(19) United States
(12) Patent Application Publication
(10) Pub. No.: US 2025/0090577 A1
(43) Pub. Date: Mar. 20, 2025

(54) CLAY MINERAL BIOMATERIAL, AND PREPARATION METHOD AND USE THEREOF

(71) Applicant: CHINA UNIVERSITY OF GEOSCIENCES (WUHAN), Wuhan (CN)

(72) Inventors: Huaming YANG, Wuhan (CN); Qianqian LIU, Wuhan (CN); Hao WANG, Wuhan (CN)

(73) Assignee: CHINA UNIVERSITY OF GEOSCIENCES (WUHAN), Wuhan (CN)

(21) Appl. No.: 18/624,133

(22) Filed: Apr. 2, 2024

(30) Foreign Application Priority Data
Sep. 14, 2023 (CN) 202311195550.9

(51) Int. Cl. A61K 35/02; A61K 33/32; A61P 29/00

(52) U.S. Cl. CPC A61K 35/02; A61K 33/32; A61P 29/00

(57) ABSTRACT

A method for preparing a clay mineral biomaterial includes: mixing montmorillonite and a potassium permanganate solution to obtain a reaction solution; subjecting the reaction solution to a hydrothermal reaction; and washing and drying a product obtained after the hydrothermal reaction to obtain the clay mineral biomaterial. In the method, montmorillonite is used as a carrier, and nano-scale manganese dioxide particles are allowed to grow in situ on the montmorillonite carrier through a hydrothermal reaction. The nano-scale manganese dioxide particles are well dispersed and fixed on a surface of the montmorillonite carrier, which can prevent the nanoparticles from aggregating. In addition, because the surface of the montmorillonite carrier is hydrophilic and rich in hydroxyl groups, there is excellent interfacial bonding between the surface of the montmorillonite carrier and the nano-scale manganese dioxide particles, and thus the montmorillonite carrier can improve the superoxide dismutase (SOD)-like and catalase (CAT)-like activities.

### CROSS-REFERENCE TO RELATED APPLICATIONS

This application claims priority to Chinese Patent Application No. 202311195550.9 with a filing date of Sep. 14, 2023. The content of the aforementioned application, including any intervening amendments thereto, is incorporated herein by reference.

### TECHNICAL FIELD

The present application belongs to the technical field of anti-inflammatory drugs, and specifically relates to a clay mineral biomaterial, and a preparation method and use thereof.

### BACKGROUND

Inflammation is a natural physiological response in an organism to protect the organism from an external damage or an internal threat. Inflammation is a normal response of a body's immune system to an external stimulus, but a long-term or excessive inflammatory response may cause a tissue damage, an immune system disorder, and the development of various diseases. In addition, an inflammation treatment is closely related to the reduction of a reactive oxygen species level. Reactive oxygen species, including free radicals and oxides, are key factors in an inflammatory process. Reactive oxygen species can damage cell membranes, DNA, and proteins to further promote the aggravation of inflammation. The reduction of a reactive oxygen species level has become an important strategy for controlling inflammation. In the treatment of inflammatory diseases, some drugs and treatment methods also focus on the regulation of a reactive oxygen species level to alleviate an inflammatory response. However, simple, economical, and effective drugs are still required to reduce a reactive oxygen species level and treat inflammation.

### SUMMARY OF PRESENT INVENTION

An objective of the present application is to provide a clay mineral biomaterial, and a preparation method and use thereof. The preparation method of the clay mineral biomaterial of the present application is very simple and cost-effective, and the clay mineral biomaterial has an excellent anti-inflammatory effect.

In one aspect, the present application provides a preparation method of a clay mineral biomaterial, including the following steps:
mixing montmorillonite and a potassium permanganate solution to obtain a reaction solution;
subjecting the reaction solution to a hydrothermal reaction; and
washing and drying a product obtained after the hydrothermal reaction to obtain the clay mineral biomaterial.

In one embodiment, the montmorillonite refers to montmorillonite refined by ball-milling.

In one embodiment, the ball-milling is conducted as follows: adding absolute ethanol to the montmorillonite, and conducting wet ball-milling at a rotational speed of 350 r/min for 60 h.

In one embodiment, after the wet ball-milling, ball-milled montmorillonite is vacuum-lyophilized.

In one embodiment, a mass ratio of the montmorillonite to potassium permanganate in the reaction solution is (50-500) mg: 1.45 g.

In one embodiment, the hydrothermal reaction is conducted at 160° C. for 48 h.

In one embodiment, the montmorillonite is medical-grade montmorillonite.

In another aspect, the present application provides a clay mineral biomaterial, where the clay mineral biomaterial is prepared by the preparation method described above, and the clay mineral biomaterial includes a montmorillonite carrier and nano-scale manganese dioxide loaded on the montmorillonite carrier.

In yet another aspect, the present application provides a use of a clay mineral biomaterial in preparation of an anti-inflammatory drug, where the clay mineral biomaterial is prepared by the preparation method described above, and the clay mineral biomaterial includes a montmorillonite carrier and nano-scale manganese dioxide loaded on the montmorillonite carrier.

Compared with the prior art, the present application has the following technical effects.

In the preparation method of the clay mineral biomaterial provided by the present application, montmorillonite is used as a carrier, and nano-scale manganese dioxide particles are allowed to grow in situ on the montmorillonite carrier through a hydrothermal reaction. In the clay mineral biomaterial, the nano-scale manganese dioxide particles are well dispersed and fixed on a surface of the montmorillonite carrier, which can prevent the nanoparticles from aggregating. In addition, because the surface of the montmorillonite carrier is hydrophilic and rich in hydroxyl groups, there is excellent interfacial bonding between the surface of the montmorillonite carrier and the nano-scale manganese dioxide particles, and thus the montmorillonite carrier can improve the superoxide dismutase (SOD)-like and catalase (CAT)-like activities of the manganese dioxide to well eliminate reactive oxygen species and reduce inflammation.

In the preparation method of the clay mineral biomaterial in the present application, montmorillonite is adopted as a raw material, which is abundant and has a low cost.

The preparation method of the clay mineral biomaterial in the present application involves simple steps and easy operations, which is conducive to large-scale production.

The clay mineral montmorillonite in the clay mineral biomaterial prepared in the present application improves the SOD-like and CAT-like activities of manganese dioxide to enhance an anti-inflammatory effect of manganese dioxide, which makes the clay mineral biomaterial have a prominent anti-inflammatory function.

### BRIEF DESCRIPTION OF THE DRAWINGS

To describe the technical solutions in the embodiments of the present application clearly, the accompanying drawings required for describing the embodiments or the prior art are described briefly below. Apparently, the accompanying drawings in the following description merely show some embodiments of the present application, and those of ordinary skill in the art may still derive other accompanying drawings from these accompanying drawings without creative efforts.

FIG. 1 shows X-ray diffraction (XRD) patterns of MMT, MnO₂, MMT/MnO₂-1, MMT/MnO₂-2, and MMT/MnO₂-3 provided in Examples 1 to 5 of the present application;

FIG. 2 shows detection results of SOD activities of MnO₂, MMT/MnO₂-1, MMT/MnO₂-2, and MMT/MnO₂-3 provided in Examples 2 to 5 of the present application;

FIG. 3 shows detection results of CAT activities of MMT, MnO₂, MMT/MnO₂-1, MMT/MnO₂-2, and MMT/MnO₂-3 provided in Examples 1 to 5 of the present application; and

FIG. 4 shows in vitro anti-inflammatory effects of MMT, MnO₂, MMT/MnO₂-1, MMT/MnO₂-2, and MMT/MnO₂-3 provided in Examples 1 to 5 of the present application.

### DETAILED DESCRIPTION OF THE EMBODIMENTS

To make the to-be-solved technical problems, technical solutions, and beneficial effects of the present application clear, the present application is further described in detail below with reference to examples. It should be understood that the specific examples described herein are merely intended to explain the present application, rather than to limit the present application.

Terms in the examples of the present application are merely used to describe the specific examples, and are not intended to limit the present application. Unless otherwise specified in the context, words in a singular form, such as “a”, “the”, and “this”, in the examples and appended claims of the present application include plural forms.

The weight of related components mentioned in the specification of the examples of the present application may not only refer to the specific content of each component, but also indicate a proportional relationship between the weights of the components. Therefore, as long as the content of related components is scaled up or reduced according to the specification of the examples of the present application, results are within the scope disclosed in the specification of the examples of the present application. Specifically, the mass mentioned in the specification of the examples of the present application may be in a mass unit known in the chemical industry, such as μg, mg, g, and kg.

The medical-grade montmorillonite in the examples of the present application is a medical-grade montmorillonite product from Shanghai Aladdin Biochemical Technology Co., Ltd., with a CAS number: 1318-93-0.

### Example 1

In Example 1 of the present application, a preparation method of montmorillonite was provided, including the following steps: An appropriate amount of medical-grade montmorillonite was weighed, absolute ethanol was added, and wet ball-milling was conducted at a rotational speed of 350 r/min for 60 h. After the wet ball-milling was completed, a ball-milling tank was taken out, a screen mesh was used to separate milling balls from a resulting mixed slurry, and the mixed slurry was vacuum-lyophilized to obtain ultra-fine montmorillonite, which was denoted as MMT. In this example of the present application, the medical-grade montmorillonite, a zirconia ball, and the absolute ethanol were in a mass ratio of 2:120:4, and the zirconia ball had a diameter of 2 mm.

### Example 2

In Example 2 of the present application, a preparation method of manganese dioxide was provided, including the following steps: 1.45 g of KMnO₄ (potassium permanganate) was added to 35 mL of H₂O to obtain a reaction solution. The reaction solution was placed in a reactor and subjected to a hydrothermal reaction at 160° C. for 48 h to obtain a manganese dioxide precipitate. The manganese dioxide precipitate was washed and lyophilized to obtain a final product, which was denoted as MnO₂.

### Example 3

In Example 3 of the present application, a preparation method of a clay mineral biomaterial was provided, including the following steps: 1.45 g of KMnO₄ (potassium permanganate) and 50 mg of the ultra-fine montmorillonite prepared in Example 1 were added to 35 mL of H₂O to obtain a reaction solution. The reaction solution was transferred to a reactor and subjected to a hydrothermal reaction at 160° C. for 48 h, such that manganese dioxide was produced on montmorillonite to obtain a precipitate. The precipitate was washed with deionized water and lyophilized to obtain a final product montmorillonite/manganese dioxide, which was denoted as MMT/MnO₂-1.

The use of the ultra-fine montmorillonite as a carrier can prolong a residence time of montmorillonite in an animal, which is conducive to the treatment of a drug including the clay mineral biomaterial for inflammation in the animal.

### Example 4

In Example 4 of the present application, a preparation method of a clay mineral biomaterial was provided, including the following steps: 1.45 g of KMnO₄ (potassium permanganate) and 100 mg of the ultra-fine montmorillonite prepared in Example 1 were added to 35 mL of H₂O to obtain a reaction solution. The reaction solution was transferred to a reactor and subjected to a hydrothermal reaction at 160° C. for 48 h, such that manganese dioxide was produced on montmorillonite to obtain a precipitate. The precipitate was washed with deionized water and lyophilized to obtain a final product montmorillonite/manganese dioxide, which was denoted as MMT/MnO₂-2.

### Example 5

In Example 5 of the present application, a preparation method of a clay mineral biomaterial was provided, including the following steps: 1.45 g of KMnO₄ (potassium permanganate) and 500 mg of the ultra-fine montmorillonite prepared in Example 1 were added to 35 mL of H₂O to obtain a reaction solution. The reaction solution was transferred to a reactor and subjected to a hydrothermal reaction at 160° C. for 48 h, such that manganese dioxide was produced on montmorillonite to obtain a precipitate. The precipitate was washed with deionized water and lyophilized to obtain a final product montmorillonite/manganese dioxide, which was denoted as MMT/MnO₂-3.

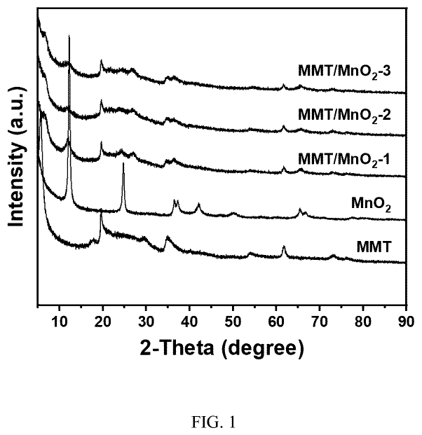

XRD patterns of MMT, MnO₂, MMT/MnO₂-1, MMT/MnO₂-2, and MMT/MnO₂-3 prepared in Examples 1 to 5 of the present application are shown in FIG. 1. It can be seen from FIG. 1 that the composite clay mineral biomaterials of montmorillonite/manganese dioxide prepared in Examples 3 to 5 of the present application each are composed of montmorillonite and manganese dioxide.

### SOD-Like Activity Assay

SOD-like activities of the MnO₂, MMT/MnO₂-1, MMT/MnO₂-2, and MMT/MnO₂-3 prepared in Examples 2 to 5 of the present application were detected with an SOD activity detection kit (Solarbio, BC0170) according to steps in instructions of the kit.

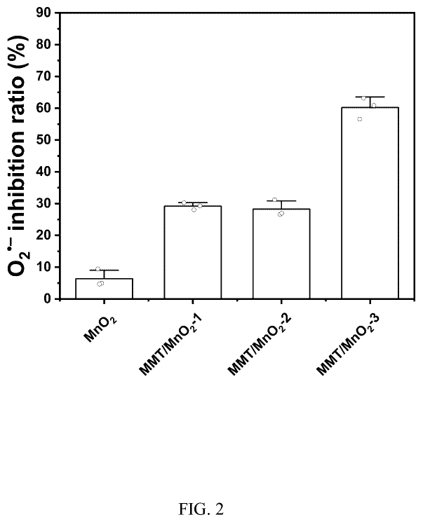

Detection results are shown in FIG. 2. O₂⁻ inhibition ratios of MMT/MnO₂-1, MMT/MnO₂-2, and MMT/MnO₂-3 are 29.20=1.10%, 28.26±2.57%, and 60.19±3.34%, respectively, and all are greater than an O₂⁻ inhibition ratio of MnO₂ (6.36±2.67%), indicating that SOD-like activities of MMT/MnO₂-1, MMT/MnO₂-2, and MMT/MnO₂-3 all are greater than an SOD-like activity of MnO₂.

### CAT-Like Activity Assay

CAT-like activities of the MMT, MnO₂, MMT/MnO₂-1, MMT/MnO₂-2, and MMT/MnO₂-3 prepared in Examples 1 to 5 of the present application were detected by detecting a content of O₂ produced from decomposition of H₂O₂ with a dissolved oxygen meter. Specifically, 8 mL of water and 100 μL of 10 M H₂O₂ or 30 μL of 1 mg/mL MMT, MnO₂, MMT/MnO₂-1, MMT/MnO₂-2, or MMT/MnO₂-3 were added to a 6-well plate, and the 6-well plate was placed for 10 min and then tested with the dissolved oxygen meter.

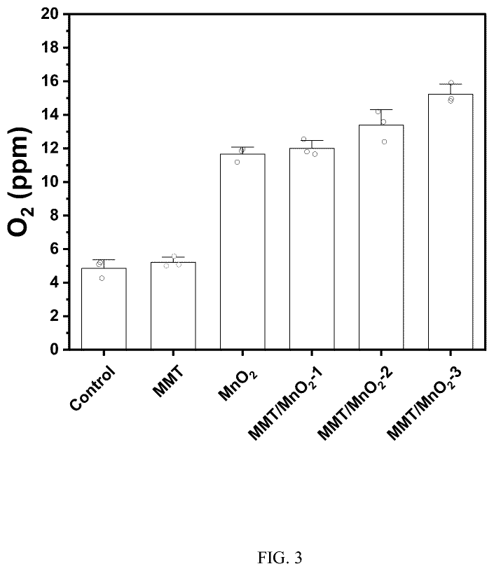

Detection results are shown in FIG. 3. O₂ productions of the composite clay mineral biomaterials MMT/MnO₂-1, MMT/MnO₂-2, and MMT/MnO₂-3 prepared in Examples 3 to 5 of the present application are 12.00±0.48 ppm, 13.39±0.92 ppm, and 15.23±0.59 ppm, respectively, and all are larger than a O₂ production of MnO₂ (11.66±0.42 ppm). A O₂ production of MMT is 5.21±0.31 ppm and is close to 4.85±0.51 ppm of a blank group. These results indicate that CAT-like activities of the composite clay mineral biomaterials prepared in Examples 3 to 5 of the present application are greater than CAT-like activities of pure manganese dioxide and pure montmorillonite.

It can be seen from FIG. 2 and FIG. 3 that, compared with MMT or MnO₂, the composite clay mineral biomaterials of montmorillonite/manganese dioxide prepared in Examples 3 to 5 of the present application are improved in terms of enzyme activities, where the MMT/MnO₂-3 exhibits the maximum enzyme activities.

### Detection of Anti-Inflammatory Effects in Cells In Vitro

In vitro anti-inflammatory effects of the MMT, MnO₂, MMT/MnO₂-1, MMT/MnO₂-2, and MMT/MnO₂-3 prepared in Examples 1 to 5 of the present application were detected. Cells RAW264.7 were coated on a 12-well plate and cultivated for 12 h, a material solution to be tested was added at a final concentration of 5 μg/mL, and the cells were further cultivated for 12 h. Then H₂O₂ was added at a final concentration of 1 mM, and the cells were further cultivated for 3 h, then washed three times with PBS, stained with a DCFH-DA stain, washed three times with PBS, and then photographed with a confocal microscope.

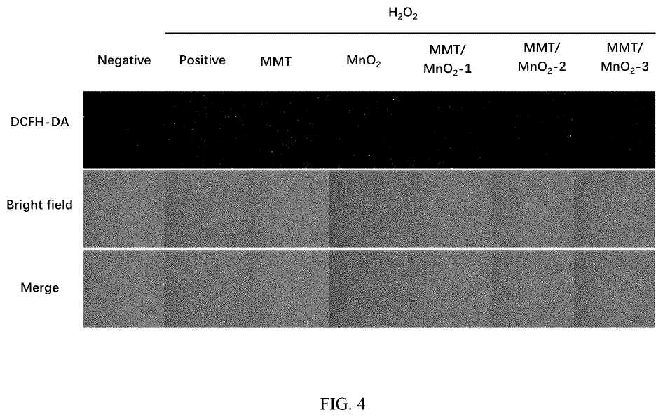

Image results are shown in FIG. 4. A quantity of cells stained green due to reactive oxygen species produced by each of the manganese dioxide-containing montmorillonite composites MMT/MnO₂-1, MMT/MnO₂-2, and MMT/MnO₂-3 prepared in Examples 3 to 5 of the present application is smaller than a quantity of cells stained green due to reactive oxygen species produced by each of MnO₂, MMT, and a blank group, and the MMT/MnO₂-3 leads to a minimum quantity of reactive oxygen species, indicating that the composite clay mineral biomaterials prepared in Examples 3 to 5 of the present application have a better anti-inflammatory effect than MMT or MnO₂.

In FIG. 4, Negative represents a negative control group in which hydrogen peroxide and a material to be tested both are not added, and Positive represents a positive control group in which hydrogen peroxide is added and a material to be tested is not added.

The above examples are merely illustrative of several implementations of the present disclosure, and the description thereof is specific and detailed, but should not be construed as a limitation to the patent scope of the present disclosure. It should be noted that those of ordinary skill in the art can further make several variations and improvements without departing from the concept of the present application, and these variations and improvements all fall within the protection scope of the present application. Therefore, the protection scope of the present application should be subject to the protection scope defined by the claims.

What is claimed is:

1. A method for preparing a clay mineral biomaterial, comprising the following steps:
mixing montmorillonite and a potassium permanganate solution to obtain a reaction solution;
subjecting the reaction solution to a hydrothermal reaction; and
washing and drying a product obtained after the hydrothermal reaction to obtain the clay mineral biomaterial.

2. The method according to claim 1, wherein the montmorillonite refers to montmorillonite refined by ball-milling.

3. The method according to claim 2, wherein the ball-milling is conducted as follows: adding absolute ethanol to the montmorillonite, and conducting wet ball-milling at a rotational speed of 350 r/min for 60 h.

4. The method according to claim 3, wherein after the wet ball-milling, ball-milled montmorillonite is vacuum-lyophilized.

5. The method according to claim 1, wherein a mass ratio of the montmorillonite to potassium permanganate in the reaction solution is (50-500) mg: 1.45 g.

6. The method according to claim 1, wherein the hydrothermal reaction is conducted at 160° C. for 48 h.

7. The method according to claim 1, wherein the montmorillonite is medical-grade montmorillonite.

8. A clay mineral biomaterial, wherein the clay mineral biomaterial is prepared by the method according to claim 1, and the clay mineral biomaterial comprises a montmorillonite carrier and nano-scale manganese dioxide loaded on the montmorillonite carrier.

9. An application of a clay mineral biomaterial in preparation of an anti-inflammatory drug, comprising: preparing the clay mineral biomaterial by the method according to claim 1, and using the clay mineral biomaterial to prepare the anti-inflammatory drug; wherein the clay mineral biomaterial comprises a montmorillonite carrier and nano-scale manganese dioxide loaded on the montmorillonite carrier.
